(19) United States
(12) Patent Application Publication
(10) Pub. No.: US 2006/0115842 A1
(43) Pub. Date: Jun. 1, 2006

(54) Highly sensitive homogeneous assay based on anti-Stokes' shift FRET measurement

(75) Inventor: Ville Laitala, Turku (FI)

Correspondence Address: BUCHANAN INGERSOLL PC;(INCLUDING BURNS, DOANE, SWECKER & MATHIS), POST OFFICE BOX 1404, ALEXANDRIA, VA, 22313-1404, US

(73) Assignee: WALLAC OY, Turku (FI)

(21) Appl. No.: 11/254,792

(22) Filed: Oct. 21, 2005

Related U.S. Application Data
(60) Provisional application No. 60/620,970, filed on Oct. 22, 2004.
(60) Provisional application No. 60/644,988, filed on Jan. 21, 2005.

(51) Int. Cl. C12Q 1/68; C07F 5/00; C07H 21/04

(52) U.S. Cl. 435/6; 534/15; 536/24.3

(57) ABSTRACT

This invention relates to a novel time-resolved lanthanide-based energy transfer assay utilizing non-overlapping acceptor fluorophores, having their absorption maximum energetically at higher level than the main emittive energy level of the donor. In this assay the lifetime of the energy transfer enhanced acceptor signal is partially independent of the energy transfer rate. The use of non-overlapping acceptors enables an anti-Stokes' shift FRET measurement, in which the acceptor emission is created and measured at a shorter wavelength than the actual donor emission.

### FIELD OF THE INVENTION

The present invention relates to an improvement in energy transfer based bioanalytical assay technology using lanthanide chelates as labels. The improvements relate to the use of non-overlapping acceptor fluorophores, which have their absorption maximum energetically at a higher level than the main emittive transitions of the donor. According to the invention, the use of non-overlapping acceptors enables anti-Stokes' shift FRET measurement, in which the FRET enhanced acceptor emission is measured at a shorter wavelength than the actual donor emission.

### BACKGROUND OF THE INVENTION

Methods based on fluorescence resonance energy transfer (FRET) have found numerous applications in the field of basic research, diagnostic assays and bioscreening. In general, the FRET technique is associated with Förster type energy transfer, in which a fluorescent donor transfers energy via non-radiative dipole-dipole interaction to an acceptor molecule (which can be fluorescent or non-fluorescent) in close proximity.¹ The Förster type energy transfer was initially introduced in the late 1940's and the theory, together with the related mathematical equations, has since been well characterized and commonly accepted.² A number of publications have been written on the Förster's theory and FRET based applications.³⁻⁷ The main requirements for the Förster mechanism include a spectral overlap between the emission of the donor and the absorption of the acceptor, which has been considered as a rule of thumb when selecting suitable donor-acceptor (D-A) pairs for FRET measurements. Further, it is well known that in the case of long lifetime donors and short lifetime (fluorescent) acceptors (τ_{D}>>τ_{A}) the lifetime of the FRET enhanced acceptor emission is determined as a function of the donor lifetime and energy transfer efficiency.⁸ This feature enables time-resolved energy transfer (TR-FRET) measurements, when micro to millisecond lifetime donors (e.g. lanthanides) are used together with regular organic fluorophores as acceptors.

The time-resolved acceptor emission monitoring is a very sensitive method to measure FRET, because in ideal circumstances the acceptor signal is specific only to the energy transfer (acceptors excited due to energy transfer). The problem related to donor emission measurements is that the donor signal always contains at least two different populations (donors that participate the energy transfer and free donors), which can not be directly resolved. Acceptor fluorescence monitoring also helps to avoid problems related to the incomplete labelling of the FRET probes since the presence of both the donor and the acceptor is required for detectable signal. Bioanalytical FRET assays based on traditional spectrally overlapping D-A pairs enable efficient energy transfer and induced acceptor emission based measurements. However, due to the overlap principle the donor always emits certain background at the acceptor measurement wavelength, and the acceptor measurement sensitivity is decreased by the donor fluorescence.

Some so called non-FRET energy transfer methods have also been published. These methods utilize acceptor molecules, which do not essentially, or at all, have an absorption overlapping with the donor emission spectrum. For example, commercial non-fluorescent labels Dabcyl and QSY 7™ have been shown to efficiently quench donor fluorescence without having a complete spectral overlap with the donor emissions.^{9;10}

U.S. Pat. No. 5,998,146 discloses an energy transfer based bioaffinity assay wherein the lanthanide energy emission and the acceptor energy absorption do not overlap. In this publication the acceptors are used as quenchers and the donor signal is measured in a time-resolved assay in the microsecond time scale.

U.S. Pat. No. 6,150,097 discloses hybridization probes that are labeled with a non-FRET pair consisting of organic fluorophore donors (ns-decay time) and a chromophore acceptor (fluorescent or non-fluorescent). In this publication the acceptors are used as quenchers and the change in the donor signal is measured.

These non-FRET methods are related to the fluorescence quenching assays (FQA), in which the energy transfer based donor emission quenching is measured. The use of non-overlapping acceptors expands the number of usable acceptor molecules in these assays but does not solve the problems related to the donor background.

### SUMMARY OF THE INVENTION

The main object of the present invention is to provide an improved time-resolved energy transfer assay utilizing at least one lanthanide donor and at least one non-overlapping acceptor fluorophore, wherein an energy transfer signal from said acceptor is measured, and wherein said acceptor emission occurs at a shorter wavelength than donor emission. The lifespan of said acceptor signal is not a direct function of the total energy transfer efficiency and luminescent decay time of the donor. Energy transfer from the different upper excited energy levels of the donor generates different decay populations to the energy transfer enhanced acceptor signal.

In a preferred embodiment the assay is based on antibody recognition reaction, receptor-ligand binding, protein binding, DNA-hybridization, DNA-cleavage or peptide cleavage. Most preferred embodiment includes homogeneous assays and multianalyte assays. Multianalyte assays may be based on one or more donor combined with more than one acceptor.

It is a further object of the present invention to provide donor-acceptor pairs useful in assays according to the present invention. Most preferred donors are lanthanide chelates, which may be luminescent or non-luminescent. Most preferred acceptors are fluorescent compounds, such as organic fluorophores.

### BRIEF DESCRIPTION OF THE DRAWINGS

FIG. 1 shows the structure of the Eu-chelate phosphoramidite block.

FIG. 2 is a normalized emission spectrum of the Eu-donor and the excitation and emission spectra of Alexa Fluor 546 acceptor.

FIG. 3. FRET using Alexa Fluor 546 as acceptor. In close proximity to the donor Alexa Fluor 546 emits strong energy transfer signal with two-exponential decay time.

FIG. 4. Normalized Eu-donor emission (7) and Alexa Fluor absorptions. (1) Alexa Fluor 488, (2) Alexa Fluor 514, (3) Alexa Fluor 532, (4) Alexa Fluor 555, (5) Alexa Fluor 546 and (6) Alexa Fluor 647.

FIG. 5. Decay curves for different acceptor emission signals using Eu-donor. Curves from top to bottom: Alexa Fluor 546 (open square), Alexa Fluor 555 (open diamond), Alexa Fluor 532 (dashed line), Alexa Fluor 514 (asterisk), Alexa Fluor 488 (open circle) and Alexa Fluor 647 (line).

FIG. 6. Simplified diagram of Eu energy levels and acceptor absorptions. For acceptors, the black dot corresponds to the wavelength of the absorption maximum and the half width of the absorption spectrum is illustrated with the error bars.

FIG. 7. Dilution curve for homogeneous ΔF508 DNA-assay using Eu-donor and Alexa Fluor 546 acceptor. Detection limit 2.5 pM.

FIGS. 8A and 8B shows the principle for dual analyte detection using the non-overlapping energy transfer method of the invention.

FIG. 9. Normalized Sm-donor emission (5) and Alexa Fluor absorbtion spectra. (1) Alexa Fluor 488, (2) Alexa Fluor 514, (3) Alexa Fluor 532 and (4) Alexa Fluor 647.

FIG. 10 shows decay curves for different acceptor emission signals using Sm-donor. (1) Alexa Fluor 532, (2) Alexa Fluor 514, (3) Alexa Fluor 488 and (4) Alexa Fluor 647.

FIG. 11. Simplified diagram of Sm energy levels and acceptor absorptions. For acceptors, the black dots correspond to the wavelength of the absorption maximum and the half width of the absorption spectrum is illustrated with the error bars.

FIG. 12. Dilution curve for the homogeneous ΔF508 DNA-assay using Sm-donor and Alexa Fluor 532 acceptor. Detection limit 11.2 pM.

### DETAILED DESCRIPTION OF THE INVENTION

It has now surprisingly been found that by using non-overlapping donor-acceptor pairs it is possible to use non-FRET measurements in bioanalytical assays, wherein the detection is based on the acceptor signal and the induced acceptor emission is measured at a shorter wavelength than the donor emission spectrum. Such an assay according to the present invention has increased sensitivity due to reduced background signal.

The main object of the present invention is thus to provide an improved time-resolved lanthanide-based energy transfer assay utilizing non-overlapping donor-acceptor pairs, which enable an anti-Stokes' shift FRET measurement, in which the acceptor emission is created and measured at a shorter wavelength than the actual donor emission.

The term “luminescence” as used herein shall cover fluorescence, phosphorescence, chemiluminescence, bioluminescence and electro-generated luminescence, photoluminescence, radioluminescence, sonoluminescence, thermoluminescence and triboluminescence.

The term “chelate” is defined as coordination complex where the central ion coordinated with at least two coordination bonds to a single ligand (multidentate ligand). These may be named by different principles, and names like chelates, supramolecular compounds, complexes, complexones, etc. are used. Special types of chelates include macrocyclic complexes, crown ethers, cryptates, calixarenes and so on.

The terms “first group” and “second group” shall be understood to include any component such as a bioaffinity recognition component (in reactions where the distance between the groups decreases or increases, e.g. in bioaffinity reactions) or a part of a molecule or substrate (e.g. the distal ends of a peptide molecule the cleavage of which will separate the two labeled groups from each other).

The term “main emittive energy level” shall be understood as an energy level, from which most of the emittive transitions of a lanthanide ion result. For example for Eu(III)-ion the main emittive energy level is the ⁵D₀-level and the emission maximums in a typical Eu(III) emission spectrum are formed by ⁵D₀→⁷Fₓ transitions.

The term “upper energy level” or “upper excited energy level” shall be understood as an energy level lying energetically above the main emittive energy level of a lanthanide ion and being capable of accepting energy via direct excitation or from excited ligand structure. Upper energy level can also be an emittive state, but the emission intensity of these states is low and/or has such a short decay time, that the emission is normally not detected in conventional time-resolved measurement utilizing micro- to millisecond-range delay time.

The term “non-overlapping acceptor” shall be understood as an acceptor, which has its absorption maximum energetically at a higher level than the main emittive energy level of the donor.

The term “energy transfer enhanced acceptor emission” or “induced acceptor emission” shall be understood as an acceptor emission (or signal), which forms as a result of energy transfer from excited donor to the fluorescent acceptor compound.

The term “decay component” shall be understood as a single decay population in a multiexponential decay curve (e.g. compound A and B having different decay times τ_{A} and τ_{B} produce both their own decay component to the resulting decay curve when measured simultaneously).

The term “low quantum yield” shall be understood as referring to quantum yields below 1%.

The term “decay profile” shall be understood as a synonym for decay curve of a luminescent sample.

In a preferred embodiment of the present invention the assay is based on non-overlapping luminescent acceptor molecules, which have their absorption maximum energetically at a higher level than the main emittive energy level of the donor. The energy transfer efficiency is dependent on the D-A distance and close proximity of the donor and the acceptor results in energy transfer, which is obtained as energy transfer enhanced emission of the non-overlapping acceptor. The nFRET based induced acceptor emission is characterized by exceptional decay properties. The induced acceptor emission does not strictly follow the commonly accepted Förster's equations defining the relation between energy transfer efficiency and fluorescence decay time. In nFRET the energy transfer results from the excited energy levels above the main emittive energy level of the donor lanthanide.

The decay time of the induced acceptor signal is not a direct function of the energy transfer rate and the donor decay time. Further, the energy transfer from the different upper energy-levels of the lanthanide donor generates different lifetime populations to the induced acceptor signal. In an assay according to the present invention, the use of non-overlapping acceptors also enable the anti-Stokes' shift FRET measurement, in which the acceptor emission is created and measured at a shorter wavelength than the main emittive transitions of the donor. To our knowledge this is a new way to carry out energy transfer measurements.

The assay according to the present invention involves non-radiative resonance energy transfer and should be considered as FRET but in purpose to make a difference to Förster type energy transfer this system will herein be referred to as nFRET (non-overlapping FRET). Non-overlapping acceptor fluorophores are similarly referred as nFRET-acceptors. Further excited energy levels of the donor, which are energetically above the main emittive energy level of the donor, are referred as upper excited energy levels of the donor.

In a highly preferred embodiment of the present invention the donor is a chelate consisting of a ligand structure and a lanthanide metal central ion. For example ions of europium, samarium, dysprosium, terbium, neodymium, erbium or thulium lanthanide metals can be used. Lanthanide ions are characterized by their 4fⁿ electronic configuration, where the orbitals are well shielded from the environment and are minimally involved in bonding, and hence the lanthanides have well defined energetic levels. Measured energy level data for Ln(III) ions can be found from many publications. Typically lanthanides have certain emittive energy level (from which the photon emission occurs) and many other practically non-emittive energy levels above the emittive energy level. In the nFRET method according to the present invention the acceptor is selected so that it has absorbtion maximum at higher energy than the main emittive energy level of the donor, i.e. the acceptor absorbtion overlaps energetically with excited energy levels above the main emittive energy level of the donor.

Lanthanide ions can be excited directly with strong excitation light. However, the light absorbtion of lanthanides is very weak and therefore ligand structures are normally used to absorb the excitation light and further to donate the excitation energy to a certain energy level of the lanthanide ion. In the scope of the invention it is important that the upper energy levels of the donor can be excited using suitable ligand structures or direct excitation.

Suitable ligand structures for chelates according to this invention are described for example in WO98/15830 and U.S. Pat. No. 5,998,146 and references cited therein. Preferred properties of a chelate according to this invention include high stability, high absorbtivity and efficient energy transfer from ligand to the upper energy levels of the central ion. However, a high overall quantum yield of the chelate is not necessary, because energy is transferred from the upper energy levels and the main emittive energy level of the donor is not directly related to the energy transfer efficiency.

The donor of the present invention is not limited to lanthanide chelates. The donor can also be an up-converting phosphor (also called up-converting chelates), which consist of certain lanthanide (emitter) embedded in a crystalline host lattice (absorber). The up-converting phosphor can act as donor according to the present invention, when the acceptor is selected so that it has absorbtion maximum at higher energy than the main emittive energy level of the lanthanide emitter. Up-converting phosphors have been described for example in U.S. Pat. No. 5891656 and their use in traditional homogeneous energy-transfer assays has been described for example in WO 2004086049. Up-converting phosphors are typically excited at wavelengths over 600 nm, and show luminescence, which is characteristic for the lanthanide emitter.

Further, in a preferred embodiment of the present invention the acceptor is an organic fluorophore, such as Alexa Fluor 546, or inorganic crystal, such as CdSe, CdS and CdTe semiconductor crystals. Preferred properties of the acceptor include short fluorescent decay time (τ<1 μs), high quantum yield, sharp emission spectrum, short Stokes' shift, high absorbtion coefficient and the capability for easy attachment of the acceptor to an assay component. For example, a suitable acceptor for the present invention can be chosen from the different groups described in WO98/15830 and U.S. Pat. No. 5,998,146 and references cited therein, such as xanthene dyes, carbocyanine dyes, squaraine dyes and porphyrins. Semiconductor crystals have been described in detail in recent publications.

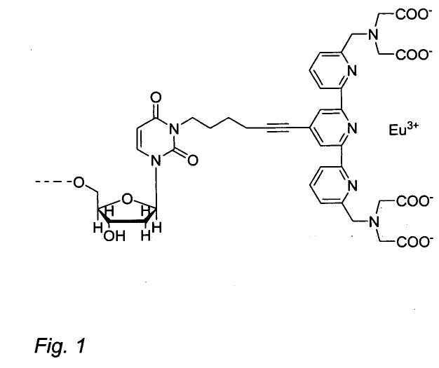

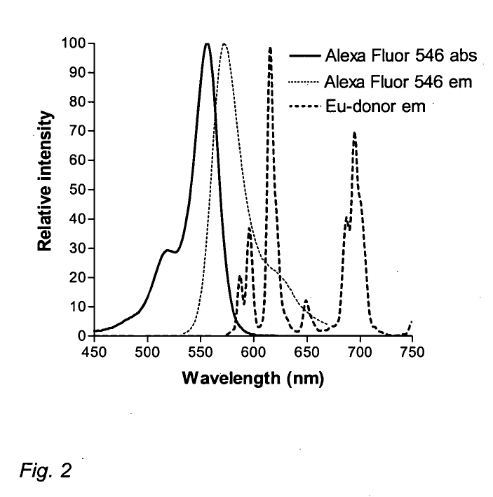

According to the present invention the acceptor is selected so that the absorbtion maximum of the acceptor is energetically at a higher level than the emission spectrum of the donor lanthanide chelate, i.e. the acceptor absorbtion maximum overlaps energetically with the excited energy levels above the main emittive energy level of the lanthanide chelate. The proof of principle for nFRET mechanism and nFRET based assay according to the present invention is described in detail in Example 1, where Eu-donor- and Alexa Fluor 546-labeled DNA-probes hybridize to a specific target-DNA to form an energy transfer complex. The structure of the Eu-donor, Eu-terpyridine chelate attached to a modified nucleotide, is shown in FIG. 1. The normalized emission spectra of the Eu-donor together with the excitation and emission spectra of the Alexa Fluor 546 acceptor are shown in FIG. 2. Alexa Fluor 546 has only a minor spectral overlap with the ⁵D₀→⁷F₀ transition of the Eu-donor. In the assay the hybridization of the donor- and acceptor-labeled probes with the target-DNA brings the labels in close proximity and results in strong induced acceptor emission, which proves the non-overlapping energy transfer scheme correct.

Due to the spectral scheme of the present invention also the emission maximum of the acceptor is at higher energy than the emission spectrum of the donor, when acceptors with relatively short Stokes' shift (e.g. traditional organic fluorophores) are used as nFRET acceptors. Such a spectral scheme of non-overlapping energy transfer enables the measurement of acceptor signal using wavelengths, which are blue-shifted compared to the donor emission spectrum, called anti-Stokes' shift FRET measurement. This is new aspect in energy transfer measurements and in anti-Stokes' shift FRET measurement the donor emission crosstalk in the acceptor channel can be remarkably reduced. In traditional Förster type measurements utilizing spectrally overlapping label pairs the donor emission crosstalk in the acceptor emission channel always limits the sensitivity of the measurement. As shown in FIG. 2 the emission maximum of the Alexa Fluor 546 is located at shorter wavelength than the ⁵D₀→⁷F₀ transition of the Eu-donor. In the measurement (Example 1) a bandpass filter 572/7 nm (bandwidth 7 nm) was used to measure nFRET induced acceptor emission, i.e. a wavelength below the emission spectrum of the Eu-donor. However the anti-Stokes' shift FRET measurement is not necessary for nFRET acceptor and the nFRET induced acceptor emission can be measured using any appropriate wavelength.

The use of lanthanide donors provides, as such, improved sensitivity compared to the use of the organic donor fluorophores, because of the TR-FRET principle and narrow emission lines of the lanthanides. Anti-Stokes' shift FRET measurement provides an additional way to suppress the donor background.

The nFRET method of the invention is useful with any lanthanide donor, which has excited energy levels above their main emittive energy level. For example, an Sm-terpyridine chelate used with nFRET acceptors gives a strong energy transfer, Example 6.

A further object of the present invention is thus to provide donor-acceptor pairs useful in assays according to the present invention, wherein the donor-acceptor pairs are chosen according to the principles described above.

The energy level scheme of the nFRET assay according to the present invention produces induced acceptor emission, which has new kind of decay properties as compared to the traditional Förster type (overlapping) energy transfer. In nFRET the energy transfer results from the upper excited energy levels of the donor, and each of these levels can produce their own decay component to the induced acceptor signal, i.e. the decay of the induced acceptor signal is not only a function of the total energy transfer efficiency and donor decay time, but is also related to the number and properties of the different energy levels, from which the energy is transferred. The decay of the induced acceptor emission can be multi-exponential if two or more upper excited energy levels are participating simultaneously to the energy transfer (each energy transfer process produces one decay component to the induced acceptor signal). This is a new aspect energy transfer based acceptor emission measurements. In Example 2 the ⁵D₂ and ⁵D₁ excited levels of the Eu-donor produce short and long decay components to the induced acceptor signal, respectively. With Sm-donor the induced nFRET signal is single exponential (Example 5), which indicates that probably only one upper excited level of the Sm-ion is participating to the energy transfer. It is also possible that the additional energy transfer processes are too fast to be detected with the current instrumentation.

The decay behavior of the nFRET induced acceptor signal can be utilized in basic time-resolved measurements, i.e. in lifetime based assays and in time-gated detection based assays. In addition the decay behavior can be utilized in multianalyte applications. As shown in Example 2, the decay profile of the induced acceptor emission is partially determined by which excited upper energy levels of the donor overlap energetically with the acceptor absorbtion. Multianalyte assays may be carried out using the same donor chelate for all analytes and analyte specific nFRET acceptors, which have different kind of energetic overlap with upper excited energy levels of the donor. Energy transfer from different excited upper energy levels results in divergent decays for different acceptors, even if the D-A distance is identical in different analyte specific energy transfer complexes. Multianalyte assays may be carried out using time-gated measurement or fluorescence lifetime measurement for different analytes. As shown in Example 4 two-analyte assay can be carried out for example using Eu-donor for both analytes and specific Alexa Fluor 514 and 546 labeled probes for different analytes. Alexa Fluor 514 accepts energy only from ⁵D₂ energy level and shows single exponential and short decay signal. Alexa Fluor 546 accepts energy from both ⁵D₂ and ⁵D₁ energy levels and shows two-exponential signal, which has short and long decay components. The Alexa Fluor 546 signal may be measured without Alexa Fluor 514 crosstalk using appropriate delay time in time-gated measurement. Alexa Fluor 546 crosstalk in the time-gated Alexa Fluor 514 measurement window can be avoided using appropriate optical filtering.

The nFRET technique according to the present invention is also useful in other multianalyte applications. Basically there are two ways to make energy transfer based multianalyte assays. One option is to use different label pair (e.g. Eu and Tb with suitable acceptors) for each analyte. Another option is to use one generic donor and several acceptors, which overlap with the same or different emittive transitions of the lanthanide donor (e.g. ⁵D₀→⁷F₂ and ⁵D₀→⁷F₄). The number of analytes and suitable acceptors can further be increased using non-overlapping acceptors, i.e. the nFRET technique can be integrated with traditional FRET technique.

A highly preferred assay format according to the present invention is based on the use of nucleic acid probes labeled with donor-acceptor pairs according to the present invention. However, the nFRET method of the present invention is equally useful in other assay formats, either in assays where dissociation is to be followed, or in association based assays where complex formation is to be followed, i.e. change a change in the label distance is to be followed. Examples of such other assay formats include, but are not limited to, assays based on antibody recognition reaction, protein binding, receptor-ligand binding, DNA-hybridization, DNA-cleavage and peptide cleavage. The scope of this invention is intended to include such formats.

Thus, the present invention provides a novel energy transfer assay utilizing luminescent nFRET acceptors, which have their absorption maximum energetically above the main emittive energy level of the donor. The assay is characterized in that it produces energy transfer enhanced acceptor emission, which is not following the Förster's theory. The spectral scheme of the nFRET also enables the anti-Stokes' shift FRET measurement, in which the acceptor emission occurs at shorter wavelength than the donor emission spectrum. This results in very low donor background in the acceptor measurement and improves detection sensitivity. It is suggested that in non-overlap case the energy transfer arises from the upper excited energy levels of the lanthanide donor. This assumption is supported by the correlation of acceptor emission behavior with the simplified energy level scheme of the lanthanide donors and the acceptors.

Assays according to the present invention have potential for carrying out new high sensitivity homogenous assays, and are useful in clinical diagnostics and other bioassays requiring high sensitivity.

The following examples are given to further illustrate preferred embodiments of the present invention, but are not intended to limit the scope of the invention. It will be obvious to a person skilled in the art, as the technology advances, the inventive concept can be implemented in various ways. The invention and its embodiments are not limited to the examples described above but may vary within the scope of the claims.

### EXAMPLES

### Example 1

This example demonstrates the proof of principle for nFRET and anti-stokes' shift FRET measurement.

A homogeneous model assay was developed for synthetic ΔF508 (cystic fibrosis) mutated DNA-target 5′-TTAAAGAAAATATCATTGGTGTTTCC TATGATGAATATAGATACAGAAGCGTCA-3′. Mutant and wild-type target specific donor-probe (3′-TACTTATATCTATGTCTTC-5′) was labeled to its 3′ end with Eu-terpyridine chelate W8044 (PerkinElmer, Wallac, Finland). ΔF508 mutation specific acceptor-probe (3′-AAATTATAGTAACCACAAA-5) was labeled to its 5′ end with Alexa Fluor 546 dye (Molecular Probes, USA). The underlined letters in the sequence denote bases that are noncomplementary to the target-sequence and prevent the probe from acting as primer during PCR. Hybridization was performed in room temperature in a total volume of 200 μl containing 15 mM Tris-HCl (pH 8), 2.5 mM MgCl₂, 50 mM KCl, 100 mM NaCl and 0.1% TritonX-100. The hybridized samples were dispensed to microtitration plates (1508-0010, PerkinElmer, Wallac), 25 μl per well in 4 replicates. Time-resolved measurements were made on a laboratory build TR-fluorometer utilizing nitrogen laser (model 79111, Oriel, USA, <10 ns pulse width, 45 Hz), photomultiplier tube (Hamamatsu, Japan) and Turbo MCS multichannel scaler (EG&G, USA) with 0.1 μs time resolution. Energy transfer based emission was collected through 572/7 nm emission filter (band width 7 nm, Omega Optical USA).

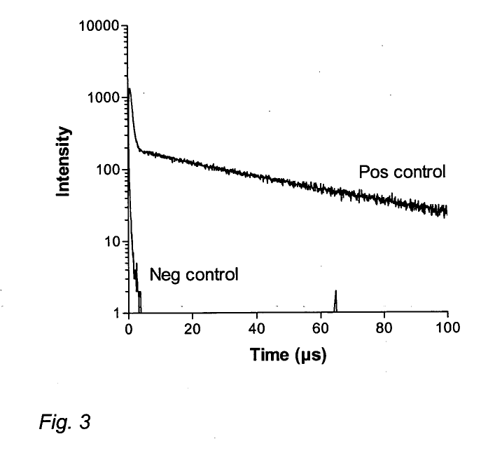

Alexa Fluor 546 has only a minor spectral overlap with the ⁵D₀→⁷F₀ transition of europium, as shown in FIG. 2. Additionally, the second peak at 596 nm is associated to ⁵D₀→⁷F₁ transition, which is defined to a magnetic dipole transition and cannot participate to Förster type energy transfer over a long distance. The nFRET was measured by comparing the acceptor emission signals between a positive sample (1 nM DNA-target+ten-fold excess of donor- and acceptor-probes) and a negative control (10 nM donor- and acceptor-probes). The fluorescence decay curves for the samples are shown in FIG. 3. As opposed to negative control, the positive sample produced very strong Alexa Fluor 546 signal with relatively long lifetime as a result of nFRET. The decay data for the energy transfer was best fitted two-exponentially having decay components of 0.64 μs and 48.4 μs. The decay data confirms that the measured Alexa Fluor 546 signal is energy transfer enhanced and not due to direct excitation of acceptor molecules. The natural lifetime of Alexa Fluor 546 is <10 ns but the long decay time of the Eu-donor (τ_{D}=1169 μs, data not shown) allows the induced acceptor emission to occur in the microsecond time range.

In the developed nFRET assay the induced acceptor emission occurs at shorter wavelength than the donor emission spectrum and allows the measurement of acceptor signal using a wavelength, which is blue-shifted as compared to the donor emission spectrum. This is new feature in energy transfer assays and the phenomenon is called anti-Stokes' shift FRET measurement. In this example the emission maximum of Alexa Fluor 546 is at a shorter wavelength than all the radiative ⁵D₀→⁷Fₓ transitions of Eu³⁺ (FIG. 2) and we used a narrow bandpass filter at 572/7 nm, i.e. a wavelength band below the donor emission, to measure nFRET.

### Example 2

Testing of Different nFRET Acceptors

This example demonstrates that different upper energy levels of the Eu-donor cause different decay populations to the induced nFRET acceptor signal.

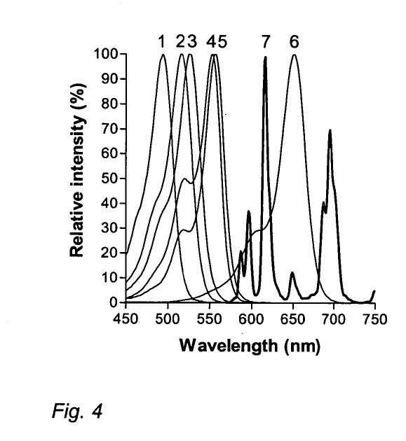

Additional series of nFRET acceptors was introduced to the same model assay as described in Example 1. The Alexa Fluors 555, 532, 514 and 488 (Molecular Probes), used as acceptors, each have slightly different absorption properties and are spectrally more blue-shifted than Alexa Fluor 546 (FIG. 4). A reference decay curve of Förster type energy transfer was measured using Alexa Fluor 647 acceptor (Molecular Probes), which has strong spectral overlap with the Eu-donor (FIG. 4). In the plate fluorometer we used bandpass filter 530/7 nm for Alexa Fluors 488 and 514, and 572/7 nm for Alexa Fluors 532, 546 and 555. Alexa Fluor 647 was measured using 665/7 nm bandpass filter.

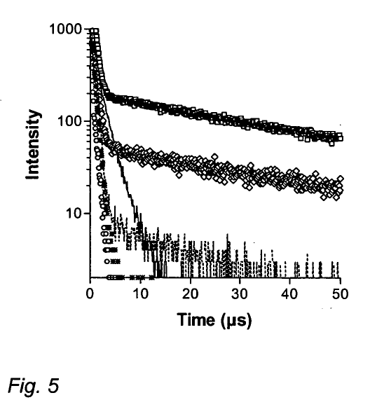

Background subtracted decay curves for different positive samples are shown in FIG. 5 and the fitted fluorescence decay times (τ_{AD}) are listed in Table 1. Both lifetime components (τ_{AD1}/τ_{AD2}) are shown for two-exponential lifetime fitting.

Every nFRET acceptor in the series emits energy transfer enhanced fluorescence and has clearly different decay response than the Förster type acceptor Alexa Fluor 647. The nFRET acceptors can be divided roughly into two categories on the basis of the decay behavior of the induced signal. Alexa Fluors 488 and 514 have single-exponential decay (short decay component only) whereas Alexa Fluors 532, 546 and 555 have two-exponential fluorescence decay (short and long decay components). The observed decay components do not correlate with the decay time of Alexa Fluor 647. Moreover, the decay times for the decay components of different nFRET acceptors are basically constant (i.e. τₛₕₒᵣₜ˜1 μs and τ_{long}˜50 μs). On the basis of this result it was calculated that energy transfer can take place both from the ⁵D₁ and ⁵D₂ energy levels of Eu and the obtained decay components are related to the decay times of these energy levels.

TABLE 1

| Acceptor | τ_{AD} (μs) |
|---|---|
| Alexa Fluor 488 | 0.63 |
| Alexa Fluor 514 | 0.66 |
| Alexa Fluor 532 | 0.56 / 31.3 |
| Alexa Fluor 546 | 0.64 / 48.4 |
| Alexa Fluor 555 | 0.69 / 54.0 |
| Alexa Fluor 647 | 1.9 |

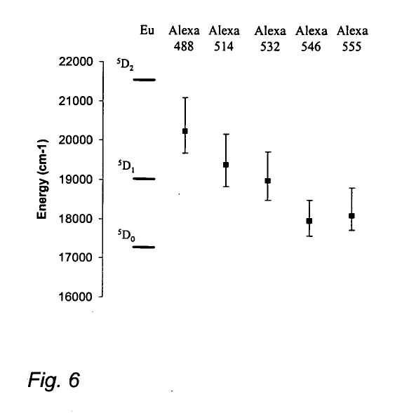

A semi-quantitative energy level scheme (FIG. 6) was used to study energetic properties of the Eu-donor and nFRET acceptors. In FIG. 6 the acceptor singlet levels are shown together with the half width of their absorption spectrum, because the energetic levels of organic fluorophores are not as well defined as line-type energy levels of lanthanide ions. A good correlation can be found between the energy level scheme and the experimental results. Alexa Fluors 488 and 514 are to the most part located above the ⁵D₁ energy level but below ⁵D₂ energy level and can therefore accept energy only from ⁵D₂ level (single-exponential decay). Alexa Fluors 555 and 546 lie below the ⁵D₂ and ⁵D₁ (but above ⁵D₀) energy levels, and can accept energy both from the ⁵D₂ and ⁵D₁ energetic levels, which correlates with the two-exponential decay. The singlet level of Alexa Fluor 532 is at almost equal level with the ⁵D₁ and it also seems to have an intermediate form of energy transfer signal (two-exponential, but with mixed decay times) when compared to other nFRET acceptors. Energy levels higher than the ⁵D₂ were not considered as potential energy donating levels, because the triplet state of the ligand lies at 22500 cm^{-1 11} and only lanthanide energy levels below the ligand triplet state can accept energy from the ligand.

As a conclusion the decay time of the induced nFRET acceptor signal is dependent on the donor energy level, from which the energy transfer occurs. The decay of the induced signal can be adjusted with the energetic properties of nFRET acceptor and nFRET produces signal, which have different kind of decay behavior than Förster type energy transfer.

### Example 3

This example demonstrates that the decay time and decay profile of the nFRET induced acceptor signal is not a direct function of the total energy transfer efficiency and donor decay time.

In Förster type energy transfer the total energy transfer efficiency is determined by the equation
E=1−τ_{DA}/τ_{D}=1−τ_{AD}/τ_{D} [1]

where τ_{D} is the decay time of the free donor, τ_{DA} is the decay time of the donor in the presence of acceptor and τ_{AD} is the decay time of the energy transfer induced acceptor emission. The latter part of the equation is valid only when τ_{D}>>τ_{A} (τ_{D}=1169 μs and τ_{A}<10 ns for the labels of this example).

The direct comparison of energy transfer efficiencies with different nFRET acceptors is difficult based on the measured acceptor signals, because the selected wavelength bands, filter transmittance differences and different quantum yields of the acceptors can have significant contribution on the measured signal intensity. The total energy transfer efficiency was determined for the nFRET acceptor series described in Example 2 by measuring the total donor quenching Qₜₒₜ during energy transfer, Table 2. Theoretical decay time for induced acceptor signal was calculated using equation [1], Table 2. Donor intensity measurement is independent of the parameters mentioned above and can be used to measure the total energy transfer efficiency in chosen conditions. To ensure a complete hybridization of the donor probe, we used 1 nM Eu-probe+5 nM DNA-target+20 nM acceptor-probe, and donor intensity was measured using bandpass filter 700/10 nm to avoid any residual interference of acceptor emission in the gated detection using a delay of 500 μs and 400 μs integration time.

TABLE 2

| Acceptor | Qₜₒₜ (%) | τ_{AD} (μs) (calc.) |
|---|---|---|
| Alexa Fluor 488 | 80.5 | 228.0 |
| Alexa Fluor 514 | 88.0 | 140.3 |
| Alexa Fluor 532 | 87.9 | 141.4 |
| Alexa Fluor 546 | 86.7 | 155.5 |
| Alexa Fluor 555 | 86.6 | 156.6 |

As shown in Table 2, all nFRET acceptors have similar quenching efficiency in the range 80-88% regardless of the differences in the decay profiles between different the acceptors. The theoretical decay time based on donor quenching of does not correlate to the measured acceptor decay (Table 1) with any of the tested nFRET acceptors. This result further proves the non-Förster behavior of the induced nFRET emission.

### Example 4

This example demonstrates the sensitivity of the nFRET technique.

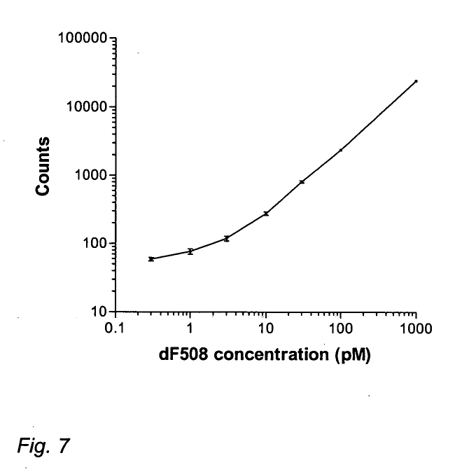

The assay described in Example 1 was used to measure a dilution curve for the mutant ΔF508 DNA-target. The homogeneous detection mix contained 10 nM donor- and acceptor-probes and the time-gated intensity was measured using 572/7 nm bandpass filter with a delay time of 10 μs and counting time of 20 μs. The assay result is shown in FIG. 7. The assay produced linear response for the analyte and a detection limit (S/B=2) of 2.5 pM was obtained. This shows that non-overlapping energy transfer is very efficient and provides high sensitivity together with the anti-Stokes' shift FRET measurement.

### Example 5

This example demonstrates how the exceptional decay time of nFRET signal can be utilized in homogeneous multianalyte assay. In this example the ΔF508 mutant- and wild type-DNA targets are detected simultaneously from the same reaction.

ΔF508 mutant and wild-type target specific donor-probe (3′-TACTTATATCTATGTCTTC-5′) is labeled to its 3′ end with Eu-terpyridine chelate W8044 (PerkinElmer, Wallac). Mutant specific acceptor probe (3′-AAATTATAGTAACCACAAA-5) is labeled to its 5′ end with Alexa Fluor 546 and wild-type specific acceptor probe (3′-ATTAGTAGAAACCACAAA-5′) is labeled to its 5′ end with Alexa Fluor 514. Wild-type ΔF508 (5′-AAGAAAATATCATCTTTGGTGTTTCCTATGATGAATATAGATACAGAAGCG TCA-3′) and mutant ΔF508 (5′-TTAAAGAAAATATCATTGGTGTTTCCTATGAT GAATATAGATACAGAAGCGTCA-3′) targets are hybridized with the detection probes in room temperature in a total volume of 200 μl containing 15 mM Tris-HCl (pH 8), 2.5 mM MgCl₂, 50 mM KCl, 100 mM NaCl and 0.1% TritonX-100. After hybridization the energy transfer signals are measured in a time resolved manner using optical channel 570/10 nm, delay 10 μs, and integration time 30 μs for Alexa Fluor 546 and optical channel 535/15 nm, delay 1 μs, and integration time 5 μs for Alexa Fluor 514.

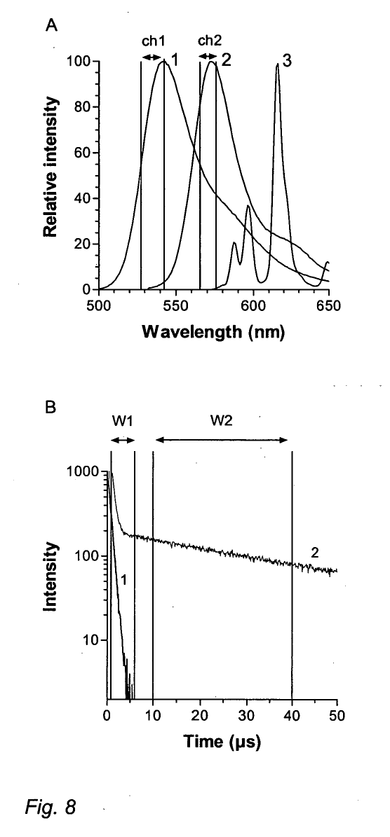

Emission spectra and the optical channels for Alexa Fluors 514 and 546 are shown in FIG. 8A. The hypothetical decay curves and time-resolved measurement windows are shown in FIG. 8B. The decay curve for the induced Alexa Fluor 546 signal is the same as in Example 1 (same DNA-probes). The decay curve for induced Alexa Fluor 514 signal in this dual-assay can be assumed to similar with the decay observed for Alexa Fluor 514-probe in Example 2. This is because the D-A distance in the hybridized sample of this assay is the same as in the hybridized sample in Example 2. Alexa Fluor 514 emits fluorescence to the optical channel of Alexa Fluor 546 (ch2) but crosstalk is avoided with appropriate time-gated measurement window (W2), FIGS. 8A and 8B. Alexa Fluor 546 crosstalk in the time-gated measurement window of the Alexa Fluor 514 (W1) is avoided using appropriate optical filtering (ch1), FIGS. 8A and 8B.

### Example 6

This example demonstrates that also other lanthanides than Eu are suitable for nFRET.

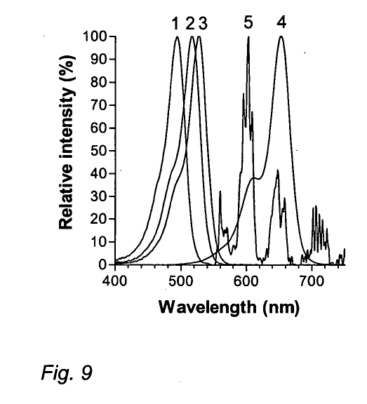

Sm-terpyridine donor was introduced to the same model assay as described in Example 1 and was tested with Alexa Fluor 532, 514 and 488 acceptors. A reference decay curve of Förster type energy transfer was measured using Alexa Fluor 647 acceptor. The energy transfer was measured by comparing the acceptor emission signals between a positive sample (1 nM DNA-target+ten-fold excess of donor- and acceptor-probes) and a negative control (10 nM donor- and acceptor-probes). In the plate fluorometer we used 530/7 nm bandpass filter for Alexa Fluor 488, 545/7 nm for Alexa Fluors 514 and 532 and 665/7 nm for Alexa Fluor 647. The spectral scheme of the D-A pairs is shown in FIG. 9. Alexa Fluors 532, 514 and 488 have negligible overlap with Sm-emission and can be considered as nFRET acceptors for Sm.

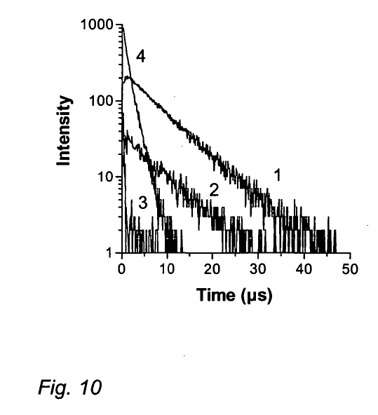

The background subtracted decay curves for the induced acceptor signals are shown in FIG. 10 and fitted decay times are shown in Table 3. All nFRET acceptors emit single-exponential energy transfer enhanced emission. The Alexa Fluor 532 and 514 signals are very strong whereas Alexa Fluor 488 signal is weakly detectable. As obtained also for Eu-donor in Example 2, the decay times of the nFRET signals differ remarkably from the decay time of the Förster type energy transfer (Alexa Fluor 647). Moreover, the decay time is nearly the same for all nFRET acceptors. The results have similar features with the results obtained using Eu-donor and show that the nFRET mechanism is also applicable with other lanthanides than Eu.

TABLE 3

| Alexa Fluor | Measured τ_{AD} (μs) |
|---|---|
| 488 | 6.6 |
| 514 | 8.0 |
| 532 | 7.6 |
| 647 | 1.3 |

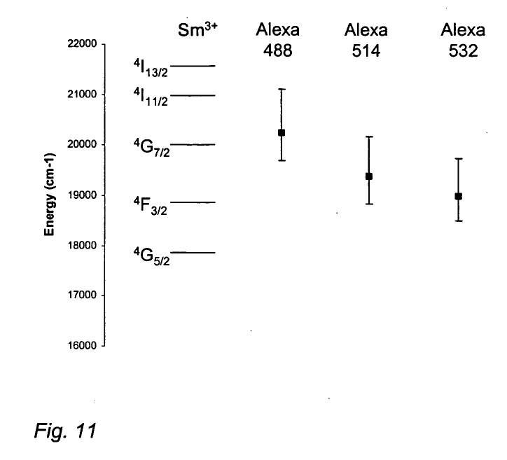

A simplified energy level scheme for Sm-donor based nFRET is shown in FIG. 11. Based on the scheme and measured results it seems that energy transfer occurs from Sm-donor to tested nFRET acceptors through ⁴G_{7/2} energy level (⁴G_{5/2} is the emittive energy level of Sm). Alexa Fluor 488 is energetically slightly above the ⁴G_{7/2} level and it produces very weak induced signal, because energy transfer is not favoured upstream. Alexa Fluors 514 and 532 lie below ⁴G_{7/2} level and produce strong single-exponential signal.

### Example 7

This example demonstrates that the sensitivity of an nFRET assay is not directly related to the quantum yield of the donor chelate.

The Sm—Alexa Fluor 532 pair was used to measure a dilution curve for the mutant ΔF508 DNA-target. The homogeneous detection mix contained 10 nM donor- and acceptor-probes and the time-gated intensity was measured using 545/7 nm bandpass filter with a delay time of 2.5 μs and counting time of 10 μs.

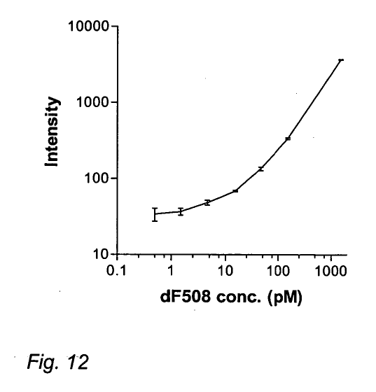

The assay result is shown in FIG. 12. The analyte response is linear and a detection limit (S/B=2) of 11.2 pM was obtained. The difference in assay sensitivity compared to the corresponding assay with Eu-terpyridine donor (Example 4.) is less than 5-fold. This is remarkable because the quantum yield of the Sm-terpyridine chelate is approximately 100-fold smaller than quantum yield of Eu-terpyridine chelate (data not shown). In the case of non-overlapping energy transfer the quantum yield of the donor is not necessary important because the energy transfer takes place from the upper excited energy levels than the emittive energy level. Efficient absorbtivity of the ligand and efficient intra-molecular energy transfer from the ligand to the upper energy levels of the central ion are the key processes in nFRET. Based on this result it is assumed that really low quantum yield lanthanides (quantum yield ˜0) can be good donor for nFRET assays.

### REFERENCE LIST

1. Förster, T. Ann. Phys. Leipzig 1948, 2, 55-75.
2. Stryer, L.; Haugland, R. P. Proc. Natl. Acad. Sci. U.S.A. 1967, 98, 719-26.
3. Clegg, R. M. Methods Enzymol. 1992, 211, 353-88.
4. Schiller, P. W. Biochemical Fluorescence Concepts, Chen, R. F.; Edelhoch, H., Eds.; Dekker: New York, 1975; Chapter 5.
5. Selvin, P. R. Methods Enzymol. 1995, 246, 300-34.
6. Stryer, L. Annu. Rev. Biochem. 1978, 47, 819-46.
7. Wu, P.; Brand, L. Anal. Biochem. 1994,1-13.
8. Morrison, L. E. Anal. Biochem. 1988,174, 101-20.
9. Karvinen, J.; Laitala, V.; Makinen, M. L.; Mulari, O.; Tamminen, J.; Hermonen, J.; Hurskainen, P.; Hemmila, I. Anal. Chem. 2004, 76, 1429-36.
10. Karvinen, J.; Elomaa, A.; Makinen, M. L.; Hakala, H.; Mukkala, V. M.; Peuralahti, J.; Hurskainen, P.; Hovinen, J.; Hemmila, I. Anal.Biochem. 2004, 325, 317-25.
11. Latva, M.; Takalo, H.; Mukkala, V. M.; Matachescu, C.; Rodrígues-Ubis, J. C.; Kankare, J. J. Luminescence 1997, 75, 149-69.

1. An improved time-resolved energy transfer based bioaffinity assay utilizing a first group labeled with at least one lanthanide donor and a second group labeled with at least one non-overlapping luminescent acceptor compound, wherein an energy transfer signal from said acceptor is measured.

2. The assay according to claim 1, wherein the lifespan of said acceptor signal is not a direct function of the total energy transfer efficiency and luminescent decay time of the donor.

3. The assay according to claim 1, wherein the energy transfer from different upper excited energy levels of the donor generates different decay populations to the energy transfer enhanced acceptor emission.

4. The assay according to claim 1, wherein said acceptor emission occurs at a shorter wavelength than the emission from the main emittive energy level of a said donor.

5. The assay according to claim 1, wherein said assay is based on nucleic acid hybridization, antigen-antibody recognition, protein binding, receptor-ligand binding, DNA-cleavage or peptide cleavage.

6. The assay according to claim 1, wherein said assay is performed as a homogeneous assay.

7. The assay according to claim 1, wherein said assay is a multianalyte assay.

8. The assay according to claim 7, wherein using a general donor for all analytes and different fluorescent acceptors for each analyte.

9. A donor-acceptor pair for use in a method according to claim 1.

10. The donor-acceptor pair according to claim 9, wherein the donor is a lanthanide chelate or up-converting phosphor, and the acceptor is a fluorophore.

11. The donor-acceptor pair according to claim 10, wherein the donor is a low quantum yield lanthanide chelate.

12. The donor-acceptor pair according to claim 10, wherein the donor is a non-luminescent lanthanide chelate.

13. The donor-acceptor pair according to claim 10, wherein the lanthanide chelate is selected from the group consisting of europium, samarium, dysprosium, terbium, neodynium, erbium and thulium chelates.

14. The donor-acceptor pair according to claim 11, wherein the fluorophore is selected from the group consisting of Alexa Fluor 546, Alexa Fluor 555, Alexa Fluor 532, Alexa Fluor 514 and Alexa Fluor 488.


SEQUENCE LISTING (8 sequences)

SEQ ID NO: 1 - DNA, 54 bp, Artificial Sequence
  mutated DNA-target sequence
ttaaagaaaatatcattggtgtttcctatgatgaatatagatacagaagcgtca

SEQ ID NO: 2 - DNA, 19 bp, Artificial Sequence
  mutant and wild-type target specific donor- probesequence
tacttatatctatgtcttc

SEQ ID NO: 3 - DNA, 19 bp, Artificial Sequence
  mutation specific acceptor-probe
aaattatagtaaccacaaa

SEQ ID NO: 4 - DNA, 19 bp, Artificial Sequence
  mutant and wild-type target specific donor- probe sequence
tacttatatctatgtcttc

SEQ ID NO: 5 - DNA, 19 bp, Artificial Sequence
  mutant specific acceptor probe
aaattatagtaaccacaaa

SEQ ID NO: 6 - DNA, 19 bp, Artificial Sequence
  Alexa Fluor 546 and wild-type specific acceptor probe sequence
aattagtagaaaccacaaa

SEQ ID NO: 7 - DNA, 54 bp, Artificial Sequence
  wild-type F508
aagaaaatatcatctttggtgtttcctatgatgaatatagatacagaagcgtca

SEQ ID NO: 8 - DNA, 54 bp, Artificial Sequence; identical to SEQ ID NO: 1
  mutant F508